(19) United States
(12) Patent Application Publication
(10) Pub. No.: US 2026/0365757 A1
(43) Pub. Date: Nov. 19, 2026

(54) MEDICINAL COMPOSITION USABLE FOR PREVENTING AND/OR TREATING BLOOD COAGULATION FACTOR IX ABNORMALITY, COMPRISING MULTISPECIFIC ANTIGEN BINDING MOLECULE REPLACING FUNCTION OF BLOOD COAGULATION FACTOR VIII

(71) Applicants: PUBLIC UNIVERSITY CORPORATION NARA MEDICAL UNIVERSITY, Nara (JP); CHUGAI SEIYAKU KABUSHIKI KAISHA, Tokyo (JP)

(72) Inventors: Midori Shima, Nara (JP); Keiji Nogami, Nara (JP); Kenichi Ogiwara, Nara (JP)

(73) Assignees: PUBLIC UNIVERSITY CORPORATION NARA MEDICAL UNIVERSITY, Nara (JP); CHUGAI SEIYAKU KABUSHIKI KAISHA, Tokyo (JP)

(21) Appl. No.: 19/573,470

(22) Filed: Mar. 20, 2026

Related U.S. Application Data
(63) Continuation of application No. 18/472,949, filed on Sep. 22, 2023, now Pat. No. 12,600,798.
Application No. 18/472,949 is a division of application No. 16/496,089, filed on Mar. 30, 2018, now abandoned.

(30) Foreign Application Priority Data
Mar. 31, 2017 (JP) 2017-069714

(51) Int. Cl. C07K 16/36

(52) U.S. Cl. CPC C07K 16/36; C07K 2317/31; C07K 2317/565; C07K 2317/75

(57) ABSTRACT

The present inventors examined the procoagulant activity of a multispecific antigen-binding molecule that functionally substitutes for FVIII using blood and plasma derived from FIX disorder patients. The result showed that multispecific antigen-binding molecules that functionally substitute for FVIII can be used not only as methods for preventing and/or treating bleeding in hemophilia A, acquired hemophilia A, von Willebrand disease, and hemophilia C, which are caused by FVIII dysfunction, but also as methods for preventing and/or treating bleeding in FIX disorders, because of their procoagulant activity. Furthermore, the effect of a FIX formulation could be enhanced by using it in combination with a multispecific antigen-binding molecule that functionally substitutes for FVIII, and it was shown that the combined use is promising as a combination therapy that shows stable hemostatic effects.

### CROSS-REFERENCE TO RELATED APPLICATIONS

This application is a continuation of U.S. application Ser. No. 18/472,949, filed on Sep. 22, 2023, which is a divisional of U.S. application Ser. No. 16/496,089, filed on Sep. 20, 2019 (abandoned), which is the National Stage of International Application No. PCT/JP2018/013547, filed on Mar. 30, 2018, which claims the benefit of Japanese Application No. 2017-069714, filed on Mar. 31, 2017. The entire content of parent application Ser. No. 18/472,949 is hereby incorporated by reference.

### SEQUENCE LISTING

This application contains a Sequence Listing that has been submitted electronically as an XML file named 14875-0269003_SL_ST26.xml. The XML file, created on Sep. 19, 2023, is 23,247 bytes in size. The material in the XML file is hereby incorporated by reference in its entirety.

### TECHNICAL FIELD

The present invention relates to pharmaceutical compositions for use in the prevention (prophylaxis) and/or treatment of blood coagulation factor IX (FIX) disorder, which comprise a multispecific antigen-binding molecule that substitutes for the function of (functionally substitute for) blood coagulation factor VIII (FVIII).

### BACKGROUND ART

FIX disorder is a rare bleeding disorder caused by a congenital defect or dysfunction of FIX (NPL 1). FIX disorder is also called hemophilia B. FIX is an enzyme precursor, and when the blood coagulation reaction commences, it changes from the precursor to activated blood coagulation factor IX (FIXa) having enzyme activity. FIXa forms a blood coagulation factor X-activating complex (tenase complex) together with an activated blood coagulation factor VIII (FVIIIa), and plays an important role in promoting blood coagulation reaction through activation of blood coagulation factor X (FX). Similarly, FVIII disorder is a hemorrhagic disease caused by congenital defect or dysfunction of FVIII, and is also called hemophilia A. In FIX disorder, activated partial thromboplastin time (APTT) is abnormally prolonged, similarly to FVIII disorder. FVIII disorder and FIX disorder are distinguished by quantifying FVIII and FIX (FVIII:C and FIX:C for activity quantification, or FVIII:Ag and FIX:Ag for factor antigen quantification). Bleeding in FIX disorder is mainly treated by FIX formulations, and regular replacement therapy in which a FIX formulation is administered on a regular basis to prevent bleeding (preventive administration) is also carried out. At present, the most commonly used regular replacement therapy protocol for FIX disorder (hemophilia B) is intravenous administration of 25 IU/kg to 40 IU/kg FIX formulation twice a week. However, many different protocols are performed even within the same country, and studying an optimal protocol is a future issue (NPL 1). Furthermore, recently in Japan, FIX formulations with prolonged half-life have been developed, and regular replacement therapy in which intravenous administration is carried out at 50 IU/kg once a week or at 100 IU/kg once every ten days has also become available (NPL 2). However, repeated intravenous administration has the problem of difficulty in securing vascular access, and is very burdensome to the patients and care givers particularly in pediatric cases (NPL 3).

Hemophilia is a hemorrhagic disease caused by congenital defect or dysfunction of FVIII or FIX among the blood coagulation factors. The disease caused by FVIII abnormality is called hemophilia A and the disease caused by FIX abnormality is called hemophilia B. Severity of the bleeding symptom of hemophilia correlates well with the activity level of the deficient coagulation factor in the blood. Patients with activity of less than 1% are classified as severe, patients with activity of 1% or more and less than 5% are classified as moderate, and patients with activity of 5% or more and less than 40% are classified as mild. Patients with severe symptoms, accounting for approximately half of hemophilia patients, exhibit bleeding symptoms several times a month. This frequency is remarkably high compared to moderate patients and mild patients. Therefore, in patients with severe hemophilia, therapy of replacing the deficient coagulation factor (FVIII or FIX) to maintain the activity of the coagulation factor in the blood at 1% or more is considered to be effective for preventing development of bleeding symptoms (NPL 1).

For prevention and/or treatment of bleeding in hemophilia patients, coagulation factor formulations (hemophilia A: FVIII; hemophilia B: FIX) purified from plasma or prepared by genetic engineering techniques are mainly used. These formulations are used for on-demand administration to stop bleeding when it occurs, or for preventive administration to prevent development of bleeding events (NPLs 1 and 4). However, FVIII formulations have a blood half-life of approximately 12 hours, and they therefore need to be administered as frequently as approximately three times a week for the preventive administration (NPLs 5 and 6). The half-life of FIX formulations in blood is 16 hours to 28 hours, and is also reportedly largely different between individuals, ranging from three hours to 38 hours. Thus, for the preventive administration, administering a FIX formulation as frequently as approximately twice a week is a standard protocol (NPLs 5, 6, and 7). In the on-demand administration, formulations must be additionally administered at regular intervals, when necessary, to prevent re-bleeding. In addition, the coagulation factor formulations are administered intravenously. Therefore, there is a strong need for a pharmaceutical agent the administration of which is less burdensome (less frequent administration, and no need for intravenous injection) than the existing coagulation factor formulations.

Moreover, antibodies against the administered coagulation factor formulations (inhibitors) may develop in hemophilia patients (NPL 8). There are reports that for hemophilia A, inhibitors develop in 20% to 30% of severe patients, while for hemophilia B, the development of inhibitors is less frequent than hemophilia A, but occurs in 1% to 6% (NPLs 8 and 9). Such inhibitors cancel the effects of the supplied coagulation factor formulations, making subsequent prevention/treatment with the coagulation factor formulations difficult. For bleeding in patients who have developed inhibitors (inhibitor patients), bypassing agents (activated blood coagulation factor VII (FVII) formulations or APCC formulations) are administered (NPLs 1 and 8). Their mechanisms of action are less dependent on the function of FVIII formulations or FIX formulations, and may show hemostatic actions even for hemophilia in the presence of inhibitors. However, because of their short half-life of approximately two hours to eight hours in blood, frequent intravenous injections are necessary, and their hemostatic activity is not enough to fully stop bleeding in some cases. Therefore, there is a strong need for a pharmaceutical agent which is not affected by the presence of inhibitors and the administration of which is less burdensome.

Acquired hemophilia, in which blood coagulation factors become impaired due to acquired development of autologous neutralizing antibodies against blood coagulation factors, may be a related bleeding disorder (NPLs 10 and 11). In most cases, acquired hemophilia is caused by development of anti-FVIII autoantibodies. Bypass formulations and such are administered for bleeding in patients with such acquired bleeding disorders; however, similarly, the problem is that frequent intravenous injections are necessary and there are cases where their hemostatic activity is not enough to fully stop bleeding.

In addition, as bleeding abnormality in which FVIII is involved, von Willebrand disease (von Willebrand's disease) caused by functional abnormality or deficiency of von Willebrand factor (vWF) is known. vWF is not only necessary for platelets to undergo normal adhesion to the subendothelial tissues at lesion sites of vascular walls, but it is also necessary for forming a complex with FVIII and keeping FVIII in the blood at a normal level. In von Willebrand disease patients, these functions are decreased, leading to hemostasis dysfunction (NPL 12). For bleeding in von Willebrand disease patients, FVIII preparations containing desmopressin (DDAVP) or plasma-derived vWF are administered, but similarly, the problem is that frequent intravenous injections are necessary and there are cases where their hemostatic activity is not enough to fully stop bleeding.

Recently, multispecific antigen-binding molecules which recognize both FIXa and FX, and have a function that substitutes for the cofactor function of FVIII, specifically, the function of promoting the activation of FX by FIXa, have been found (PTLs 1, 2, and 3). Among the multispecific antigen-binding molecules, bispecific antibodies are being developed as pharmaceutical compositions for prevention and/or treatment of bleeding in hemophilia A (PTLs 1, 2, and 3, and NPLs 13, 14, 15, 16, 17, and 18). Furthermore, application of the bispecific antibodies to prevention and/or treatment of bleeding in acquired hemophilia A and von Willebrand disease, and hemophilia C, which involve dysfunction of FVIII, has also been contemplated (PTLs 1, 2, 3, and 4). In addition, use of the bispecific antibodies in combination with a FIX formulation is also contemplated in the treatment of hemophilia A (PTL 5).

### PRIOR ART REFERENCES

### Non-patent Literatures

[NPL 1] Guidelines for the management of hemophilia, 2005, World Federation of Hemophilia
[NPL 2] Alprolix for intravenous administration, package insert, 2017
[NPL 3] Haemophilia 2011; 17: 2-10
[NPL 4] Nature 1984; 312: 330-337
[NPL 5] Nature 1984; 312: 337-342
[NPL 6] Biochim Biophys Acta 1986; 871: 268-278
[NPL 7] Haemophilia 2007; 13: 663-669
[NPL 8] Blood 2007; 109(2): 546-551
[NPL 9] Haemophilia 2013; 19: 2-10
[NPL 10] Semin Thromb Hemost 2012; 38: 433-446
[NPL 11] Thromb Haemost 2013; 110: 1114-1120
[NPL 12] Blood 2013; 122: 3735-3740
[NPL 13] Nature Medicine 2012; 18(10): 1570-1574
[NPL 14] PLOS ONE 2013; 8(2): e57479
[NPL 15] J Thromb Haemost 2014; 12(2): 206-213
[NPL 16] Blood 2014; 124(20): 3165-3171
[NPL 17] Blood 2016; 127(13): 1633-1641
[NPL 18] N Engl J Med 2016; 374: 2044-2053

### Patent Literatures

[PTL 1] WO 2005/035756
[PTL 2] WO 2006/109592
[PTL 3] WO 2012/067176
[PTL 4] WO 2016/171202
[PTL 5] WO 2016/166014

### SUMMARY OF INVENTION

### Problems to be Solved by the Invention

An objective of the present invention is to provide pharmaceutical compositions for use in the prevention and/or treatment of FIX disorder, which comprise multispecific antigen-binding molecules that substitute for the function of (functionally substitute for) FVIII.

### Means for Solving the Problems

In order to solve the above problems, the present inventors examined the blood/plasma coagulation-promoting action (procoagulant activity) of “multispecific antigen-binding molecules substituting for the function of FVIII (FVIII function-substituting molecules)” by coagulation evaluation methods, ROTEM and APTT, using commercially-available FIX-deficient human plasma and blood/plasma derived from patients with FIX disorder.

As a result, the present inventors discovered that in each of the above-described coagulation evaluation methods, multispecific antigen-binding molecules that functionally substitute for FVIII show procoagulant activity in blood/plasma derived from FIX disorder patients and in FIX-deficient human plasma. Therefore, multispecific antigen-binding molecules that substitute for the function of FVIII can not only be used as prophylactic (preventive) and/or therapeutic agents against bleeding in hemophilia A, acquired hemophilia A, von Willebrand disease and hemophilia C, which are caused by dysfunction of FVIII, but also as prophylactic and/or therapeutic agents against bleeding in FIX disorder, because of its procoagulant activity.

Based on these findings, the present invention relates to pharmaceutical compositions for use in the prophylaxis (prevention) and/or treatment of FIX disorder, excluding hemophilia A, acquired hemophilia A, von Willebrand disease and hemophilia C, comprising a multispecific antigen-binding molecule that substitutes for the function of FVIII. More specifically the present invention relates to the following:
[1] a pharmaceutical composition for use in preventing and/or treating blood coagulation factor IX disorder, which comprises a multispecific antigen-binding molecule that functionally substitutes for blood coagulation factor VIII;
[2] the pharmaceutical composition of [1], wherein the multispecific antigen-binding molecule that functionally substitutes for blood coagulation factor VIII is a bispecific antibody which recognizes blood coagulation factor IX and/or activated blood coagulation factor IX, and blood coagulation factor X;
[3] the pharmaceutical composition of [2], wherein the bispecific antibody is:
   a bispecific antibody in which a first polypeptide and a third polypeptide form a pair and a second polypeptide and a fourth polypeptide form a pair, wherein the first polypeptide consists of an H chain comprising the H chain CDR 1, 2, and 3 amino acid sequences of SEQ ID NOs: 1, 2, and 3 (H chain CDRs of Q499), the second polypeptide consists of an H chain comprising the H chain CDR 1, 2, and 3 amino acid sequences of SEQ ID NOs: 4, 5, and 6 (H chain CDRs of J327), and the third polypeptide and the fourth polypeptide consist of a common L chain comprising the L chain CDR 1, 2, and 3 amino acid sequences of SEQ ID NOs: 7, 8, and 9 (L chain CDRs of L404);
[4] the pharmaceutical composition of [2] or [3], wherein the bispecific antibody is:
   a bispecific antibody in which a first polypeptide and a third polypeptide form a pair and a second polypeptide and a fourth polypeptide form a pair, wherein the first polypeptide consists of an H chain comprising the H chain variable region amino acid sequence of SEQ ID NO: 13, the second polypeptide consists of an H chain comprising the H chain variable region amino acid sequence of SEQ ID NO: 14, and the third polypeptide and the fourth polypeptide consist of a common L chain comprising the L chain variable region amino acid sequence of SEQ ID NO: 15;
[5] the pharmaceutical composition of any one of [2] to [4], wherein the bispecific antibody is:
   a bispecific antibody in which a first polypeptide and a third polypeptide form a pair and a second polypeptide and a fourth polypeptide form a pair, wherein the first polypeptide consists of an H chain consisting of the amino acid sequence of SEQ ID NO: 10, the second polypeptide consists of an H chain consisting of the amino acid sequence of SEQ ID NO: 11, and the third polypeptide and the fourth polypeptide consist of a common L chain of SEQ ID NO: 12.
[6] The pharmaceutical composition of any one of [1] to [5], wherein the blood coagulation factor IX disorder is a disease that develops and/or progresses due to a decrease, dysfunction, and/or defect in the activity of blood coagulation factor IX and/or activated blood coagulation factor IX;
[7] the pharmaceutical composition of any one of [1] to [6], wherein the blood coagulation factor IX disorder is a congenital or acquired disease;
[8] the pharmaceutical composition of any one of [1] to [7], wherein the blood coagulation factor IX disorder is hemophilia B or blood coagulation factor IX deficiency disease;
[9] the pharmaceutical composition of any one of [1] to [8], which is for combined use with a blood coagulation factor IX formulation;
[10] the pharmaceutical composition of any one of [1] to [8], which is for enhancing the blood coagulation activity of a blood coagulation factor IX formulation;
[11] a combination medicament for use in preventing and/or treating blood coagulation factor IX disorder, which is a combination of a multispecific antigen-binding molecule that functionally substitutes for blood coagulation factor VIII and a blood coagulation factor IX formulation;
[12] a method of enhancing the blood coagulation activity of a blood coagulation factor IX formulation in a patient with a blood coagulation factor IX disorder, using a multispecific antigen-binding molecule that functionally substitutes for blood coagulation factor VIII;
[13] a blood coagulation factor IX formulation for use in combination with the pharmaceutical composition of any one of [1] to [8];
[14] a blood coagulation factor IX formulation for enhancing the FVIII function-substituting activity of the pharmaceutical composition of any one of [1] to [8];
[15] a method for enhancing the FVIII function-substituting activity of a multispecific antigen-binding molecule that functionally substitutes for blood coagulation factor VIII in a hemophilia B patient, the method using a blood coagulation factor IX formulation;
[A1] a method for preventing and/or treating a blood coagulation factor IX disorder, which comprises administering to a patient a multispecific antigen-binding molecule that functionally substitutes for blood coagulation factor VIII;
[A2] the method of [A1], wherein the multispecific antigen-binding molecule that functionally substitutes for blood coagulation factor VIII is a bispecific antibody which recognizes blood coagulation factor IX and/or activated blood coagulation factor IX, and blood coagulation factor X;
[A3] the method of [A2], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain comprising the H chain CDR 1, 2, and 3 amino acid sequences of SEQ ID NOs: 1, 2, and 3 (H chain CDRs of Q499), the second polypeptide consists of an H chain comprising the H chain CDR 1, 2, and 3 amino acid sequences of SEQ ID NOs: 4, 5, and 6 (H chain CDRs of J327), and the third polypeptide and the fourth polypeptide consist of a common L chain comprising the L chain CDR 1, 2, and 3 amino acid sequences of SEQ ID NOs: 7, 8, and 9 (L chain CDRs of L404);
[A4] the method of [A2] or [A3], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain comprising the H chain variable region amino acid sequence of SEQ ID NO: 13, the second polypeptide consists of an H chain comprising the H chain variable region amino acid sequence of SEQ ID NO: 14, and the third polypeptide and the fourth polypeptide consist of a common L chain comprising the L chain variable region amino acid sequence of SEQ ID NO: 15;
[A5] the method of any one of [A2] to [A4], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain consisting of the amino acid sequence of SEQ ID NO: 10, the second polypeptide consists of an H chain consisting of the amino acid sequence of SEQ ID NO: 11, and the third polypeptide and the fourth polypeptide consist of a common L chain of SEQ ID NO: 12;
[A6] the method of any one of [A1] to [A5], wherein the blood coagulation factor IX disorder is a disease that develops and/or progresses due to a decrease, dysfunction, and/or defect in the activity of blood coagulation factor IX and/or activated blood coagulation factor IX;
[A7] the method of any one of [A1] to [A6], wherein the blood coagulation factor IX disorder is a congenital or acquired disease;
[A8] the method of any one of [A1] to [A7], wherein the blood coagulation factor IX disorder is hemophilia B or blood coagulation factor IX deficiency disease;
[A9] the method of any one of [A1] to [A8], which is a combination therapy with a blood coagulation factor IX formulation;
[A10] the method of any one of [A1] to [A8], which is a method for enhancing the blood coagulation activity of a blood coagulation factor IX formulation;
[A11] a method for preventing and/or treating a blood coagulation factor IX disorder, which comprises administering to a patient a multispecific antigen-binding molecule that functionally substitutes for blood coagulation factor VIII in combination with a blood coagulation factor IX formulation;
[A12] a method for enhancing the blood coagulation activity of a blood coagulation factor IX formulation, which comprises administering a multispecific antigen-binding molecule that functionally substitutes for blood coagulation factor VIII to a patient with a blood coagulation factor IX disorder, wherein the patient is subjected to administration of the blood coagulation factor IX formulation before, simultaneously with, or after administration of the multispecific antigen-binding molecule that functionally substitutes for blood coagulation factor VIII;
[A13] a method for preventing and/or treating blood coagulation factor IX disorder, which comprises administering a blood coagulation factor IX formulation to a patient, wherein the blood coagulation factor IX formulation is administered in combination with a multispecific antigen-binding molecule that functionally substitutes for blood coagulation factor VIII;
[A14] a method for enhancing the FVIII function-substituting activity of a multispecific antigen-binding molecule that functionally substitutes for blood coagulation factor VIII, which comprises administering a blood coagulation factor IX formulation to a hemophilia B patient, wherein the patient is subjected to administration of the multispecific antigen-binding molecule that functionally substitutes for blood coagulation factor VIII before, simultaneously with, or after administration of the blood coagulation factor IX formulation;
[B1] a multispecific antigen-binding molecule that functionally substitutes for blood coagulation factor VIII for use in a prevention method and/or a treatment method for a blood coagulation factor IX disorder;
[B2] the antigen-binding molecule of [B1], wherein the multispecific antigen-binding molecule that functionally substitutes for blood coagulation factor VIII is a bispecific antibody which recognizes blood coagulation factor IX and/or activated blood coagulation factor IX, and blood coagulation factor X;
[B3] the antigen-binding molecule of [B2], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain comprising the H chain CDR 1, 2, and 3 amino acid sequences of SEQ ID NOs: 1, 2, and 3 (H chain CDRs of Q499), the second polypeptide consists of an H chain comprising the H chain CDR 1, 2, and 3 amino acid sequences of SEQ ID NOs: 4, 5, and 6 (H chain CDRs of J327), and the third polypeptide and the fourth polypeptide consist of a common L chain comprising the L chain CDR 1, 2, and 3 amino acid sequences of SEQ ID NOs: 7, 8, and 9 (L chain CDRs of L404);
[B4] the antigen-binding molecule of [B2] or [B3], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain comprising the H chain variable region amino acid sequence of SEQ ID NO: 13, the second polypeptide consists of an H chain comprising the H chain variable region amino acid sequence of SEQ ID NO: 14, and the third polypeptide and the fourth polypeptide consist of a common L chain comprising the L chain variable region amino acid sequence of SEQ ID NO: 15;
[B5] the antigen-binding molecule of any one of [B2] to [B4], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain consisting of the amino acid sequence of SEQ ID NO: 10, the second polypeptide consists of an H chain consisting of the amino acid sequence of SEQ ID NO: 11, and the third polypeptide and the fourth polypeptide consist of a common L chain of SEQ ID NO: 12;
[B6] the antigen-binding molecule of any one of [B1] to [B5], wherein the blood coagulation factor IX disorder is a disease that develops and/or progresses due to a decrease, dysfunction, and/or defect in the activity of blood coagulation factor IX and/or activated blood coagulation factor IX;
[B7] the antigen-binding molecule of any one of [B1] to [B6], wherein the blood coagulation factor IX disorder is a congenital or acquired disease;
[B8] the antigen-binding molecule of any one of [B1] to [B7], wherein the blood coagulation factor IX disorder is hemophilia B or blood coagulation factor IX deficiency disease;
[B9] the antigen-binding molecule of any one of [B1] to [B8], wherein the prevention method and/or the treatment method is a combination therapy with a blood coagulation factor IX formulation;
[B10] the antigen-binding molecule of any one of [B1] to [B8], wherein the prevention method and/or the treatment method is a method for use in enhancing the blood coagulation activity of a blood coagulation factor IX formulation;
[B11] a combination medicament of a multispecific antigen-binding molecule that functionally substitutes for blood coagulation factor VIII and a blood coagulation factor IX formulation, which is for use in a prevention method and/or treatment method for a blood coagulation factor IX disorder;
[B12] a multispecific antigen-binding molecule that functionally substitutes for blood coagulation factor VIII, which is for use in enhancing the blood coagulation activity of a blood coagulation factor IX formulation in a patient with a blood coagulation factor IX disorder;
[B13] a blood coagulation factor IX formulation for use in a prevention method and/or a treatment method for a blood coagulation factor IX disorder, wherein the formulation is administered in combination with the antigen-binding molecule of any one of [B1] to [B8];
[B14] a blood coagulation factor IX formulation for use in enhancing the FVIII function-substituting activity of the antigen-binding molecule of any one of [B1] to [B8];
[B15] a blood coagulation factor IX formulation for use in enhancing the FVIII function-substituting activity of a multispecific antigen-binding molecule that functionally substitutes for blood coagulation factor VIII in a hemophilia B patient;
[C1] use of a multispecific antigen-binding molecule that functionally substitutes for blood coagulation factor VIII in the manufacture of a medicament for preventing and/or treating a blood coagulation factor IX disorder;
[C2] the use of [C1], wherein the multispecific antigen-binding molecule that functionally substitutes for blood coagulation factor VIII is a bispecific antibody which recognizes blood coagulation factor IX and/or activated blood coagulation factor IX, and blood coagulation factor X;
[C3] the use of [C2], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain comprising the H chain CDR 1, 2, and 3 amino acid sequences of SEQ ID NOs: 1, 2, and 3 (H chain CDRs of Q499), the second polypeptide consists of an H chain comprising the H chain CDR 1, 2, and 3 amino acid sequences of SEQ ID NOs: 4, 5, and 6 (H chain CDRs of J327), and the third polypeptide and the fourth polypeptide consist of a common L chain comprising the L chain CDR 1, 2, and 3 amino acid sequences of SEQ ID NOs: 7, 8, and 9 (L chain CDRs of L404);
[C4] the use of [C2] or [C3], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain comprising the H chain variable region amino acid sequence of SEQ ID NO: 13, the second polypeptide consists of an H chain comprising the H chain variable region amino acid sequence of SEQ ID NO: 14, and the third polypeptide and the fourth polypeptide consist of a common L chain comprising the L chain variable region amino acid sequence of SEQ ID NO: 15;
[C5] the use of any one of [C2] to [C4], wherein the bispecific antibody is the following antibody:
   a bispecific antibody in which a first polypeptide and a third polypeptide form a pair and a second polypeptide and a fourth polypeptide form a pair, wherein the first polypeptide consists of an H chain consisting of the amino acid sequence of SEQ ID NO: 10, the second polypeptide consists of an H chain consisting of the amino acid sequence of SEQ ID NO: 11, and the third polypeptide and the fourth polypeptide consist of a common L chain of SEQ ID NO: 12;
[C6] the use of any one of [C1] to [C5], wherein the blood coagulation factor IX disorder is a disease that develops and/or progresses due to a decrease, dysfunction, and/or defect in the activity of blood coagulation factor IX and/or activated blood coagulation factor IX;
[C7] the use of any one of [C1] to [C6], wherein the blood coagulation factor IX disorder is a congenital or acquired disease;
[C8] the use of any one of [C1] to [C7], wherein the blood coagulation factor IX disorder is hemophilia B or blood coagulation factor IX deficiency disease;
[C9] the use of any one of [C1] to [C8], wherein the medicament is a combination medicament with a blood coagulation factor IX formulation;
[C10] the use of any one of [C1] to [C8], wherein the medicament is a medicament for enhancing the blood coagulation activity of a blood coagulation factor IX formulation;
[C11] use of a multispecific antigen-binding molecule that functionally substitutes for blood coagulation factor VIII and a blood coagulation factor IX formulation in the manufacture of a combination medicament for preventing and/or treating a blood coagulation factor IX disorder;
[C12] use of a multispecific antigen-binding molecule that functionally substitutes for blood coagulation factor VIII in the manufacture of a medicament for enhancing the blood coagulation activity of a blood coagulation factor IX formulation in a patient with a blood coagulation factor IX disorder;
[C13] use of a blood coagulation factor IX formulation in the manufacture of a medicament for preventing and/or treating a blood coagulation factor IX disorder, which is a combination medicament with a multispecific antigen-binding molecule that functionally substitutes for blood coagulation factor VIII; and
[C14] use of a blood coagulation factor IX formulation in the manufacture of a medicament for enhancing the FVIII function-substituting activity of a multispecific antigen-binding molecule that functionally substitutes for blood coagulation factor VIII in a hemophilia B patient.

### Effects of the Invention

Pharmaceutical compositions for use in preventing and/or treating FIX disorders other than hemophilia A, acquired hemophilia A, von Willebrand disease, and hemophilia C, the compositions comprising a multispecific antigen-binding molecule that functionally substitutes for FVIII, are provided by the present invention.

### BRIEF DESCRIPTION OF THE DRAWINGS

FIG. 1 shows the shortening ratios of blood coagulation (initiation) time according to Ca-triggered whole blood coagulability test (ROTEM) using blood samples from FIX disorder patients under regular administration therapy.

FIG. 2 shows the ratio of APTT shortening when ACE910 (100 μg/mL) was added ex vivo to plasma from FIX disorder patients. The abbreviations in the figure are explained below.
ACE: ACE910
sFIXd: commercially available FIX-deficient human plasma (Sysmex)
CogtrN: commercially available standard human plasma (Coagtrol N, Sysmex)
FIX:C: FIX activity level
FIX:Ag: FIX antigen level

FIG. 3-1 shows evaluation of the ratio of APTT shortening determined from values before and after addition of 100 μg/mL ACE910, when a FIX formulation (rFIX, BeneFix, Pfizer) at a concentration of 0, 0.01, 0.1, 1, 10, or 100 IU/dL, or an anti-FIX-Gla antibody (anti-FIX Ab) was added to a commercially available FIX-deficient human plasma (FIXd-plasma or sFIXd) and this was subjected to further ex vivo addition of ACE910.

FIG. 3-2 shows the result obtained by further adding an anti-FIX-Gla antibody (aFIXAb) to the FIX-deficient human plasma ex vivo.

### MODE FOR CARRYING OUT THE INVENTION

The multispecific antigen-binding molecule that substitutes for the function of FVIII of the present invention can also be referred to as a multispecific antigen-binding molecule having FVIII-like activity. In the present invention, the phrase “functionally substitute/substituting for FVIII (substitute/substituting for the function of FVIII)” means that FX activation by FIXa is promoted (FXa generation by FIXa is promoted). More specifically, in the present invention, the phrase “functionally substitute/substituting for FVIII (substitute/substituting for the function of FVIII)” means recognizing FIX and/or FIXa, and FX, and promoting activation of FX by FIXa (promoting FXa generation by FIXa). The activity of promoting FXa generation can be evaluated by methods well known in the art, for example using a measurement system comprising FIXa, FX, synthetic substrate S-2222 (synthetic substrate of FXa), and phospholipids. In more detail, evaluations can be carried out according to the methods described in WO 2005/035756, WO 2006/109592, WO 2012/067176, or such.

Multispecific antigen-binding molecules of the present invention comprise a first antigen-binding site and a second antigen-binding site that can specifically bind to at least two different types of antigens or epitopes. While the first antigen-binding site and the second antigen-binding site are not particularly limited as long as they have an activity to bind to FIX and/or FIXa, and FX, respectively, examples include sites necessary for binding with antigens, such as antibodies, scaffold molecules (antibody-like molecules) or peptides, or fragments containing such sites. Scaffold molecules are molecules that exhibit function by binding to target molecules, and any polypeptide may be used as long as they are conformationally stable polypeptides that can bind to at least one target antigen. Examples of such polypeptides include antibody variable regions, fibronectin (WO 2002/032925), protein A domain (WO 1995/001937), LDL receptor A domain (WO 2004/044011, WO 2005/040229), ankyrin (WO 2002/020565), and such, and also molecules described in documents by Nygren et al. (Current Opinion in Structural Biology 1997; 7:463-469; and Journal of Immunol Methods 2004; 290:3-28), Binz et al. (Nature Biotech 2005; 23:1257-1266), and Hosse et al. (Protein Science 2006; 15:14-27). Furthermore, as mentioned in Curr Opin Mol Ther 2010; 12 (4): 487-95 and Drugs 2008; 68 (7): 901-12, peptide molecules that can bind to target antigens may be used.

In the present invention, multispecific antigen-binding molecules are not particularly limited as long as they are molecules that can bind to at least two different types of antigens or epitopes, but examples include polypeptides containing the above-mentioned antigen-binding sites, such as antibodies and scaffold molecules as well as their fragments, and aptamers comprising nucleic acid molecules and peptides, and they may be single molecules or multimers thereof. Preferred multispecific antigen-binding molecules include multispecific antibodies that can bind specifically to at least two different antigens. Particularly preferred examples of multispecific antibodies of the present invention include bispecific antibodies (BsAb) that can bind specifically to two different antigens (they may also be called dual specific antibodies).

In the present invention, the term “common L chain (commonly shared L chain)” refers to an L chain that respectively forms a pair with two or more different H chains, and can show binding ability to each antigen. Herein, the term “different HI chain(s)” preferably refers to H chains of antibodies against different antigens, but is not limited thereto, and also refers to H chains whose amino acid sequences are different from each other. Commonly shared L chain can be obtained, for example, according to the method described in WO 2006/109592.

In an embodiment of the present invention, when multispecific antigen-binding molecules, multispecific antibodies, or bispecific antibodies of the present invention respectively have a plurality of antibody L chains, those antibody L chains may be different from one another or they may be a common L chain.

In an embodiment of the present invention, a multispecific antigen-binding molecule is a multispecific antigen-binding molecule that recognizes FIX and/or FIXa, and FX, and functionally substitutes for FVIII; preferably a multispecific antibody that recognizes FIX and/or FIXa, and FX, and functionally substitutes for FVIII; and more preferably a bispecific antibody that recognizes FIX and/or FIXa, and FX, and functionally substitutes for FVIII. The antigen-binding molecule or the antibody of the present invention preferably comprises variable regions in an anti-FIXa antibody and variable regions in an anti-FX antibody.

In one embodiment of the present invention, the multispecific antigen-binding molecule, multispecific antibody, or bispecific antibody comprises a first polypeptide and a third polypeptide comprising an antigen-binding site that recognizes FIX and/or FIXa, and a second polypeptide and a fourth polypeptide comprising an antigen-binding site that recognizes FX. The first polypeptide and the third polypeptide, and the second polypeptide and the fourth polypeptide, include the antigen-binding site of an antibody H chain and the antigen-binding site of an antibody L chain.

For example, in the multispecific antigen-binding molecule, multispecific antibody, or bispecific antibody in the present invention, the first polypeptide and the third polypeptide contain the antigen-binding site of an H chain and an L chain, respectively, of an antibody against FIX or FIXa, and the second polypeptide and the fourth polypeptide contain the antigen-binding site of an H chain and an L chain, respectively, of an antibody against FX. In this case, the antibody L chain antigen-binding sites contained in the first polypeptide and the third polypeptide, and in the second polypeptide and the fourth polypeptide, may be the antigen-binding site of a common L chain.

In the present invention, the polypeptides comprising an antibody L chain antigen-binding site preferably contain the sequence of the whole or a part of the L chain of an antibody binding to FIX, FIXa and/or FX.

A preferred embodiment of multispecific antigen-binding molecule that functionally substitutes for FVIII in the present invention includes, for example, a bispecific antibody that recognizes FIX and/or FIXa, and FX. Such an antibody can be obtained according to methods described, for example, in WO 2005/035756, WO 2006/109592, and WO 2012/067176. The bispecific antibody of the present invention includes antibodies described in these documents.

A preferred bispecific antibody includes the antibody (ACE910: Emicizumab) below, which is a bispecific antibody described in a patent document (WO 2012/067176):
a bispecific antibody in which a first polypeptide forms a pair with a third polypeptide and a second polypeptide forms a pair with a fourth polypeptide, wherein the first polypeptide consists of an H chain comprising the amino acid sequences of H chain CDR 1, 2, and 3 of SEQ ID NOs: 1, 2, and 3 (H chain CDRs of Q499), the second polypeptide consists of an H chain comprising the amino acid sequences of H chain CDR 1, 2, and 3 of SEQ ID NOs: 4, 5, and 6 (H chain CDRs of J327), and the third and fourth polypeptides consist of a common L chain comprising the amino acid sequences of L chain CDR 1, 2, and 3 of SEQ ID NOs: 7, 8, and 9.

More specifically, the antibody is a bispecific antibody in which a first polypeptide and a third polypeptide form a pair and a second polypeptide and a fourth polypeptide form a pair, wherein the first polypeptide consists of an H chain comprising the H chain variable region amino acid sequence of SEQ ID NO: 13, the second polypeptide consists of an H chain comprising the H chain variable region amino acid sequence of SEQ ID NO: 14, and the third polypeptide and the fourth polypeptide consist of a common L chain comprising the L chain variable region amino acid sequence of SEQ ID NO: 15.

More specifically, the antibody is a bispecific antibody (Q499-z121/J327-z119/L404-k) in which a first polypeptide and a third polypeptide form a pair and a second polypeptide and a fourth polypeptide form a pair, wherein the first polypeptide consists of an H chain consisting of the amino acid sequence of SEQ ID NO: 10, the second polypeptide consists of an H chain consisting of the amino acid sequence of SEQ ID NO: 11, and the third polypeptide and the fourth polypeptide consist of a common L chain of SEQ ID NO: 12.

Amino acids contained in the amino acid sequences described in the present invention may be post-translationally modified (for example, the modification of an N-terminal glutamine into a pyroglutamic acid by pyroglutamylation is well-known to those skilled in the art). Naturally, antibodies with such post-translationally modified amino acids are included in the antibodies used in the present invention.

Herein, the term “recognize(s)” in the phrase “an antibody which recognizes antigen A”, the term “bind(s)” in the phrase “an antibody which binds to antigen A”, and the term “specifically bind(s)” in the phrase “an antibody which specifically binds to antigen A” may be used interchangeably in a broad sense.

Polypeptides in the present invention normally refer to proteins and peptides having a length of approximately ten amino acids or longer. Generally, they are biologically derived polypeptides, but are not particularly limited to such polypeptides, and may be, for example, polypeptides comprising an artificially designed sequence. Furthermore, they may be any native polypeptides, or synthetic polypeptides, recombinant polypeptides, or such. Additionally, the fragments of the above-mentioned polypeptides are also included in the polypeptides of the present invention.

The term “antibody” is used in the broadest sense, and may be monoclonal antibodies, polyclonal antibodies, dimers, multimers, multispecific antibodies (for example, bispecific antibodies), antibody derivatives, and modified antibody products (Miller K et al. J Immunol. 2003, 170(9), 4854-61) as long as they display a desired biological activity. The antibodies may be mouse antibodies, human antibodies, humanized antibodies, chimeric antibodies, or those derived from another species, or they may be artificially synthesized antibodies. The antibodies disclosed herein can be of any type (for example, IgG, IgE, IgM, IgD, and IgA), class (for example, IgG1, IgG2, IgG3, IgG4, IgAQ1 and IgA2) or subclass of immunoglobulin molecules. The immunoglobulins can be derived from any species (for example, human, mouse, or rabbit). The terms “antibody”, “immune globulin” and “immunoglobulin” are used interchangeably in a broad sense.

The term “antibody derivative” includes a portion of an antibody, preferably an antibody variable domain, or at least an antigen-binding region of an antibody. Antibody derivatives include, for example, Fab, Fab′, F(ab′)2, Fv fragments, linear antibodies, and single-chain antibodies (scFv), sc(Fv)₂, Fab₃, domain antibodies (dAb) (WO 2004/058821, WO 2003/002609), diabodies, triabodies, tetrabodies, minibodies, and multispecific antibodies formed from antibody derivatives, but are not limited thereto. Here, “Fab” is constructed from a single light chain and the CH1 domain and variable region of a single heavy chain. Furthermore, “Fv” is the smallest antibody derivative, and includes a complete antigen-recognizing region and an antigen-binding region. The antibody derivative may be, for example, a fusion between an IgG antibody and Fc. For example, one can refer to Example 2 in U.S. Pat. No. 5,641,870 specification; Zapata G et al. Protein Eng. 1995, 8(10), 1057-1062; Olafsen T et al. Protein Eng. Design & Sel. 2004, 17(4): 315-323; Holliger P et al. Nat. Biotechnol. 2005, 23 (9): 1126-36; Fischer N et al. Pathobiology. 2007, 74(1): 3-14; Shen J et al. J Immunol Methods. 2007, 318, 65-74; and Wu et al. Nat Biotechnol. 2007, 25(11), 1290-7.

Diabodies are bivalent low-molecular weight antibodies (minibodies) constructed by gene fusion (Holliger, P. et al., Proc. Natl. Acad. Sci. USA 1993; 90:6444-6448; EP 404,097; WO 93/11161). Diabodies are dimers consisting of two polypeptide chains, in which each polypeptide chain comprises an L chain variable region (VL) and an H chain variable region (VH) linked with a linker short enough to prevent association of these two domains within the same chain, for example, a linker of preferably 2 to 12 amino acids, more preferably 3 to 10 amino acids, particularly about 5 amino acids. The polypeptide chain form a dimer since the linker between the VL and VH encoded on the same polypeptide is too short to form a single chain variable region fragment. Therefore, diabodies comprise two antigen-binding sites.

A single-chain antibody or an scFv antibody fragment comprises the VH and VL regions of an antibody, and these regions exist in a single polypeptide chain. In general, an Fv polypeptide further comprises a polypeptide linker between the VH and VL regions, and this enables an scFv to form a structure necessary for antigen binding (for a review on scFvs, see Pluckthun “The Pharmacology of Monoclonal Antibodies” Vol. 113 (Rosenburg and Moore ed. (Springer Verlag, New York) pp. 269-315, 1994). In the context of the present invention, linkers are not particularly limited so long as they do not inhibit the expression of the antibody variable regions linked at their ends.

Bispecific antibodies may be produced by chemically crosslinking Fab's. Bispecific F(ab′)₂ can be produced, for example, by preparing Fab′ from an antibody, using it to produce a maleimidized Fab′ with ortho-phenylenedi-maleimide (o-PDM), and then reacting this with Fab′ prepared from another antibody to crosslink Fab's derived from different antibodies (Keler T et al. Cancer Research 1997, 57: 4008-4014). The method of chemically linking an Fab′-thionitrobenzoic acid (TNB) derivative and an antibody fragment such as Fab′-thiol (SH) is also known (Brennan M et al. Science 1985; 229:81-83).

Instead of a chemical crosslink, a leucine zipper derived from Fos and Jun may also be used. Preferential formation of heterodimers by Fos and Jun is utilized, even though they also form homodimers. Fab′ to which Fos leucine zipper is added, and another Fab′ to which Jun leucine zipper is added are expressed and prepared. Monomeric Fab′-Fos and Fab′-Jun reduced under mild conditions are mixed and reacted to form bispecific F(ab′)₂ (Kostelny S A et al. J. of Immunology, 1992, 148: 1547-53). This method can be applied not only to Fab's but also to scFvs, Fvs, and such.

Furthermore, bispecific antibodies including sc(Fv)₂ such as IgG-scFv (Protein Eng Des Sel. 2010; 23(4): 221-8) and BiTE (Drug Discov Today 2005; 15; 10(18): 1237-44), DVD-Ig (Nat Biotechnol. 2007; 25(11): 1290-7. Epub 2007 Oct. 14; and MAbs 2009; 1(4): 339-47. Epub 2009 Jul. 10), and also others (IDrugs 2010; 13: 698-700) including two-in-one antibodies (Science 2009; 20; 323(5921): 1610-4; and Immunotherapy 2009; 1(5): 749-51), Tri-Fab, tandem scFv, and diabodies are known (MAbs 2009; 1(6): 539-547). In addition, even when using molecular forms such as scFv-Fc and scaffold-Fc, bispecific antibodies can be produced efficiently by preferentially secreting a heterologous combination of Fcs (Ridgway J B et al., Protein Engineering 1996; 9: 617-621; Merchant A M et al. Nature Biotechnology 1998; 16: 677-681; WO 2006/106905; and Davis J H et al., Protein Eng Des Sel 2010; 4: 195-202).

A bispecific antibody may also be produced using a diabody. A bispecific diabody is a heterodimer of two cross-over scFv fragments. More specifically, it is produced by forming a heterodimer using VH(A)-VL(B) and VH(B)-VL(A) prepared by linking VHs and VLs derived from two kinds of antibodies, A and B, using a relatively short linker of about 5 residues (Holliger P et al. Proc Natl. Acad. Sci. USA 1993; 90:6444-6448).

The desired structure can be achieved by linking the two scFvs with a flexible and relatively long linker comprising about 15 residues (single chain diabody: Kipriyanov S M et al. J. of Molecular Biology 1999; 293:41-56), and conducting appropriate amino acid substitutions (knobs-into-holes: Zhu Z et al. Protein Science 1997; 6:781-788; VH/VL interface engineering: Igawa T et al. Protein Eng Des Sel 2010; 8:667-77).

An sc(Fv)₂ that can be produced by linking two types of scFvs with a flexible and relatively long linker, comprising about 15 residues, may also be a bispecific antibody (Mallender W D et al. J. of Biological Chemistry 1994; 269:199-206).

Examples of modified antibody products may include antibodies linked to various molecules such as polyethylene glycol (PEG). Antibodies of the present invention include such modified antibody products. The substance to be linked is not limited in the modified antibody products of the present invention. To yield such modified antibody products, chemical modifications can be made to the obtained antibodies. Such methods are already established in this field.

“Bispecific” antibodies refer to antibodies having variable regions that recognize different epitopes, where the regions are within the same antibody molecule. Bispecific antibodies may be antibodies that recognize two or more different antigens or antibodies that recognize two or more different epitopes on the same antigen. Bispecific antibodies may include not only whole antibodies but antibody derivatives. Antibodies of the present invention also include bispecific antibodies. Herein, anti-FIXa/FX bispecific antibody and bispecific antibody that recognizes FIXa and FX are used synonymously.

### Methods for Producing Genetically Engineered Antibodies

Recombinant antibodies produced by using genetic engineering techniques can be used as the antibodies. Recombinant antibodies can be obtained by cloning DNAs encoding the antibodies from hybridomas or antibody-producing cells such as sensitized lymphocytes that produce antibodies, inserting them into vectors, and then introducing them into hosts (host cells) to produce the antibodies.

The antibodies include human antibodies, mouse antibodies, and rat antibodies, and their origin is not limited. They may also be genetically modified antibodies such as chimeric antibodies and humanized antibodies.

Methods for obtaining human antibodies are known. For example, transgenic animals carrying the entire repertoire of human antibody genes can be immunized with antigens of interest to obtain human antibodies of interest (see International Publication WO 93/12227, WO 92/03918, WO 94/02602, WO 94/25585, WO 96/34096, and WO 96/33735).

Genetically modified antibodies can be produced using known methods. Specifically, for example, chimeric antibodies comprise H chain and L chain variable regions of an immunized animal antibody, and H chain and L chain constant regions of a human antibody. Chimeric antibodies can be obtained by linking DNAs encoding the variable regions of the antibody derived from the immunized animal, with DNAs encoding the constant regions of a human antibody, inserting this into an expression vector, and then introducing it into host to produce the antibodies.

Humanized antibodies are modified antibodies that are also referred to as reshaped human antibodies. A humanized antibody is constructed by transferring the complementarity determining regions (CDRs) of an antibody derived from an immunized animal to the CDRs of a human antibody. Conventional genetic recombination techniques for such purposes are known (see European Patent Application Publication No. EP 239400; International Publication No. WO 96/02576; Sato K et al., Cancer Research 1993, 53: 851-856; International Publication No. WO 99/51743).

While bispecific antibodies are not limited to those of the IgG type, for example, IgG-type bispecific antibodies can be secreted from a hybrid hybridoma (quadroma) produced by fusing two types of hybridomas that produce IgG antibodies (Milstein C. et al., Nature 1983, 305: 537-540). They can also be secreted by introducing the L chain and H chain genes constituting the two types of IgGs of interest, a total of four types of genes, into cells to co-express the genes.

In this case, by introducing suitable amino acid substitutions to the CH3 regions of the H chains, IgGs having a heterogeneous combination of H chains can be preferentially secreted (Ridgway J B et al. Protein Engineering 1996, 9:617-621; Merchant A M et al. Nature Biotechnology 1998, 16: 677-681; WO 2006/106905; Davis J H et al. Protein Eng Des Sel. 2010, 4: 195-202).

Regarding the L chains, since the diversity of L chain variable regions is lower than that of H chain variable regions, one can expect to obtain common L chain that can confer binding ability to both H chains. The antibodies of the present invention may be antibodies comprising common L chains. Bispecific IgGs can be efficiently expressed by introducing the gene of the common L chain and both H chains into cells.

### Antibody Production Methods

Antibodies of the present invention can be produced by methods known to those skilled in the art. Specifically, DNA encoding the antibody of interest is inserted into an expression vector. Insertion into an expression vector is carried out such that the expression will take place under the control of expression regulatory regions such as enhancers and promoters. Next, host cells are transformed using this expression vector to express the antibodies. Appropriate combinations of the host and expression vector can be used in this step.

Examples of the vectors include M13 series vectors, pUC series vectors, pBR322, pBluescript, and pCR-Script. In addition to these vectors, for example, pGEM-T, pDIRECT, or pT7 can also be used for the purpose of cDNA subcloning and excision.

Particularly, expression vectors are useful for using the vectors for the purpose of producing the antibody. For example, when the host is E. coli such as JM109, DH5x, HB101, or XL1-Blue, the expression vectors indispensably have a promoter that permits efficient expression in E. coli, for example, lacZ promoter (Ward et al., Nature (1989) 341, 544-546; and FASEB J (1992) 6, 2422-2427), araB promoter (Better et al., Science (1988) 240, 1041-1043), or T7 promoter. Examples of such vectors include the vectors mentioned above as well as pGEX-5X-1 (manufactured by Pharmacia), “QIAexpress system” (manufactured by QIAGEN), pEGFP, and pET (in this case, the host is preferably BL21 expressing T7 RNA polymerase).

The vectors may contain a signal sequence for polypeptide secretion. In the case of production in the periplasm of E. coli, pelB signal sequence (Lei, S. P. et al., J. Bacteriol. (1987) 169, 4397) can be used as the signal sequence for polypeptide secretion. The vectors can be transferred to the host cells using, for example, calcium chloride methods or electroporation methods.

In addition to the E. coli expression vectors, examples of the vectors for producing the antibody of the present invention include mammal-derived expression vectors (e.g., pcDNA3 (manufactured by Invitrogen Corp.), pEGF-BOS (Nucleic Acids. Res. 1990, 18(17), p5322), pEF, and pCDM8), insect cell-derived expression vectors (e.g., “Bac-to-BAC baculovairus expression system” (manufactured by GIBCO BRL), and pBacPAK8), plant-derived expression vectors (e.g., pMH1 and pMH2), animal virus-derived expression vectors (e.g., pHSV, pMV, and pAdexLcw), retrovirus-derived expression vectors (e.g., pZIPneo), yeast-derived expression vectors (e.g., “Pichia Expression Kit” (manufactured by Invitrogen Corp.), pNV11, and SP-Q01), and Bacillus subtilis-derived expression vectors (e.g., pPL608 and pKTH50).

For the purpose of expression in animal cells such as CHO cells, COS cells, or NIH3T3 cells, the vectors indispensably have a promoter necessary for intracellular expression, for example, SV40 promoter (Mulligan et al., Nature (1979) 277, 108), MMTV-LTR promoter, EF1α promoter (Mizushima et al., Nucleic Acids Res (1990) 18, 5322), CAG promoter (Gene (1991) 108, 193), or CMV promoter and, more preferably, have a gene for screening for transformed cells (e.g., a drug resistance gene that can work as a marker by a drug (neomycin, G418, etc.)). Examples of the vectors having such properties include pMAM, pDR2, pBK-RSV, pBK-CMV, pOPRSV, and pOP13.

An exemplary method intended to stably express the gene and increase the number of intracellular gene copies involves transfecting CHO cells deficient in nucleic acid synthesis pathway with vectors having a DHFR gene serving as a complement thereto (e.g., pCHOI) and using methotrexate (MTX) in the gene amplification. An exemplary method intended to transiently express the gene involves using COS cells having a gene which expresses an SV40 T antigen on their chromosomes to transform the cells with vectors having a replication origin of SV40 (pcD, etc.). A replication origin derived from polyomavirus, adenovirus, bovine papillomavirus (BPV), or the like may be used. The expression vectors for increasing the number of gene copies in a host cell system can additionally contain a selection marker such as an aminoglycoside transferase (APH) gene, a thymidine kinase (TK) gene, an E. coli xanthine guanine phosphoribosyltransferase (Ecogpt) gene, or a dihydrofolate reductase (dhfr) gene.

The antibodies of the present invention obtained by the methods described above can be isolated from inside host cells or from outside of the cells (the medium, or such), and purified to practically pure and homogeneous antibodies. The antibodies can be separated and purified by methods routinely used for separating and purifying antibodies, and the type of method is not limited. For example, the antibodies can be separated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectrofocusing, dialysis, recrystallization, and such.

The chromatographies include, for example, affinity chromatography, ion exchange chromatography, hydrophobic chromatography, gel filtration, reverse phase chromatography, and adsorption chromatography (Strategies for Protein Purification and Characterization: A Laboratory Course Manual. Ed Daniel R. Marshak et al., Cold Spring Harbor Laboratory Press, 1996). The chromatographic methods described above can be conducted using liquid-chromatography, for example, HPLC and FPLC. Columns used for affinity chromatography include protein A columns and protein G columns. Columns using protein A include, for example, Hyper D, POROS, and Sepharose FF (GE Amersham Biosciences). The present invention includes antibodies that are highly purified using these purification methods.

Pharmaceutical compositions used for therapeutic or preventive purposes of the present invention can be prepared by mixing, if necessary, with suitable pharmaceutically acceptable carriers, vehicles, and such, and then made into lyophilized formulations or solution formulations. Examples of suitable pharmaceutically acceptable carries and vehicles include sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders. They may also comprise other low-molecular-weight polypeptides, proteins such as serum albumin, gelatin, and immunoglobulins, amino acids such as glycine, glutamine, asparagine, glutamic acid, asparagic acid, methionine, arginine, and lysine, sugars and carbohydrates such as polysaccharides and monosaccharides, and sugar alcohols such as mannitol and sorbitol. When preparing an aqueous solution for injection, physiological saline and isotonic solutions comprising glucose and other adjuvants such as D-sorbitol, D-mannose, D-mannitol, and sodium chloride may be used, and if necessary, in combination with appropriate solubilizers such as alcohol (for example, ethanol), polyalcohols (such as propylene glycol and PEG), and nonionic surfactants (such as polysorbate 80, polysorbate 20, poloxamer 188, and HCO-50). By mixing hyaluronidase into the formulation, a larger fluid volume can be administered subcutaneously (Expert Opin Drug Deliv. 2007 July; 4(4): 427-40). Alternatively, the pharmaceutical compositions of the present invention may be filled into syringes in advance. Solution formulations can be prepared according to methods described in WO 2011/090088.

The pharmaceutical compositions of the present invention can be administered to patients via any suitable route. For example, patients can be treated with bolus or continuous infusion over a period of time via an intravenous, intramuscular, intraperitoneal, intracerebral, transdermal, subcutaneous, intraarticular, sublingual, intrasynovial, oral, inhalation, topical or external route. Intravenous administration or subcutaneous administration is preferred. Patients in the present application include human patients and non-human animal patients. In the present application, the term “patient” is used interchangeably with “subject” and “individual”.

The dosage is, for example, 0.001 mg/kg to 1000 mg/kg in the case of the above-mentioned bispecific antibody. The administration interval is at least one day or longer in the case of the above-mentioned bispecific antibody.

Rotation thromboelastometry (ROTEM), activated partial thromboplastin time (APTT), clot waveform analysis (CWA), thrombin generation assay (TGA), and such are widely known as methods for monitoring the drug efficacy of blood coagulation factors such as FVIII, and those skilled in the art can use them by appropriately changing/modifying these methods. These methods may be used alone or in combination, and the drug efficacy may be determined from the results of at least one method.

ROTEM is an examination method that can comprehensively evaluate the coagulability and coagulation process of blood by monitoring a thrombelastograph (change in elasticity of clotted blood). It is generally used for analyzing causes of bleeding and for determining effects of therapeutic agents. A reaction-initiating reagent such as a Ca trigger is added to a test blood, and clotting time (CT), clot formation time (CFT), and such are measured as evaluation parameters.

APTT has long been widely used as a method for monitoring the drug efficacy of FVIII formulations. APTT is a method in which an APTT reagent is added to test plasma, after which CaCl₂) is added, and the time for fibrinogen to be converted into insoluble fibrin, that is, the time until coagulation starts, is measured.

CWA is a test in which the amount of fibrin generated as the coagulation reaction progresses is measured as optical (e.g., absorbance) changes over time. In CWA, a series of coagulation reactions from the initiation stage of fibrin formation to the amplification stage of the coagulation reaction can be evaluated over time. Furthermore, as for the coagulation-initiating reagent for CWA, the APTT reagent (Thromb Haemost 2002; 87 (3): 436-41, J Thromb Haemost 2006; 4 (2): 377-84)), a reagent with a mixed solution of a low concentration of tissue factor (TF) and a blood coagulation factor XII (FXII) activator (ellagic acid) (J Thromb Haemost 2014; 12 (3): 355-62), and such reagents have been reported. The clot waveform is a waveform representing a temporal change in optical information (absorbance) related to the amount of light. The clot waveform is differentiated (first differentiation) to calculate the coagulation velocity, and the maximum coagulation velocity is used as a parameter. The coagulation velocity is differentiated (secondary differentiation) to calculate coagulation acceleration, and the maximum coagulation acceleration is used as a parameter (Haemophilia 2008; 14: 83-92, J Thromb Haemost 2014; 12 (3): 355-62).

TGA is an assay in which the amount of thrombin generated as the coagulation reaction progresses is measured as enzymatic activity over time using a fluorescent substrate for thrombin (Haemophilia 2008; 14 (suppl. 3): 83-92).

In one embodiment of the present invention, pharmaceutical compositions used for the prevention and/or treatment of an FIX disorder, which comprise a multispecific antigen-binding molecule that substitutes for the function of FVIII, are provided. FIX disorders are rare hemorrhagic diseases caused by a congenital defect or dysfunction of FIX, and include, for example, hemophilia B and FIX deficiency disease. In addition, the decreased activity or defect of FIX includes, for example, those from congenital and/or acquired causes, but is not limited thereto. The degree of decrease in FIX activity in patients compared to normal subjects is preferably less than 40% (for example, less than 40%, less than 30%, less than 20%, less than 10%), more preferably less than 10% (for example, less than 10%, less than 9%, or less than 8%, less than 7%, or less than 6%), even more preferably less than 5% (e.g., less than 5%, less than 4%, less than 3%, or less than 2%), particularly preferably less than 1%, but is not limited thereto. Methods for measuring the activity of FIX are well known to those skilled in the art (for example, “Minna ni yakudatsu ketsuyuubyou no kiso to rinsho” (Basics and clinical practice of hemophilia useful for all), Satoshi Shirahata, Iyaku Journal, 2009, etc.).

In an embodiment of the present invention, hemophilia B patients or patients with FIX deficiency disease include patients with or without inhibitors (autoantibodies against FIX), patients with a decrease or a complete lack of FIX level, and patients with a decrease or a complete lack of FIX activity, but are not particularly limited thereto.

In one aspect, hemophilia B patients or FIX deficiency disease patients are severe hemophilia B patients or severe FIX deficiency disease patients with a trace FIX activity of less than 1.0 IU/dL. In one aspect, hemophilia B patients or FIX deficiency disease patients are hemophilia B patients or FIX deficiency disease patients who do not carry inhibitors.

The terms “hemophilia B” and “FIX deficiency disease” are used interchangeably in a broad sense.

In an embodiment, the present invention provides pharmaceutical compositions for use in preventing and/or treating blood coagulation factor IX disorder, which comprise a multispecific antigen-binding molecule that functionally substitutes for FVIII, wherein the compositions are for combined use with blood coagulation factor IX formulations (FIX formulations). In the combined use, these pharmaceutical agents may be administered simultaneously or consecutively, or one may be administered first and then the other may be administered after a period of time.

In another aspect, the present invention provides pharmaceutical compositions for use in preventing and/or treating blood coagulation factor IX disorder, which comprise a multispecific antigen-binding molecule that functionally substitutes for FVIII, wherein the composition is for enhancing the blood coagulation activity of FIX formulations.

Furthermore, in another aspect, the present invention provides combination medicaments for use in preventing and/or treating blood coagulation factor IX disorder, which are a combination of a multispecific antigen-binding molecule that functionally substitutes for blood coagulation factor VIII and a FIX formulation.

In addition, in another aspect, the present invention provides methods for enhancing the blood coagulation activity of FIX formulations in patients with a blood coagulation factor IX disorder, using a multispecific antigen-binding molecule that functionally substitutes for blood coagulation factor VIII.

The phrase “enhancing blood coagulation activity” refers to, for example, shortening of APTT by administering the multispecific antigen-binding molecule by at least one second, preferably by at least three seconds or five seconds, and more preferably by at least ten seconds, compared to that before administering the multispecific antigen-binding molecule.

Furthermore, in another aspect, the present invention provides blood coagulation factor IX formulations for combined use with the pharmaceutical composition for use in preventing and/or treating blood coagulation factor IX disorder, which comprises a multispecific antigen-binding molecule that functionally substitutes for blood coagulation factor VIII.

Furthermore, the present invention provides blood coagulation factor IX formulations for enhancing the FVIII function-substituting activity of the pharmaceutical compositions for use in preventing and/or treating blood coagulation factor IX disorder, which comprise a multispecific antigen-binding molecule that functionally substitutes for blood coagulation factor VIII.

Furthermore, the present invention provides methods for enhancing the FVIII function-substituting activity of multispecific antigen-binding molecules that functionally substitute for blood coagulation factor VIII in patients with blood coagulation factor IX disorder, using a blood coagulation factor IX formulation.

The FIX formulation may be any pharmaceutical composition comprising FIX regardless of the origin and molecular form of FIX, and includes Christmassin M, Novact M, PPSB-HT, BeneFIX (nonacog alfa), Rixubis(nonacog gamma), Alprolix (eftrenonacog alfa), and Idelvion (albutrepenonacog alfa), but is not particularly limited thereto.

In one embodiment of the present invention, FIX is not limited to human-derived FIX, and may be FIX derived from humans, bovines, pigs, dogs, cats, or mice/rats. In one aspect, FIX is a human FIX, which refers to a human FIX consisting of 415 amino acid residues, which is formed by removing the N-terminal signal sequence and the pro-peptide region consisting of 46 amino acids from the immature human FIX consisting of 461 amino acid residues (SEQ ID NO: 16) (see for example, UniProtKB/Swiss-Prot Accession P00740-1). Human FIX is shown by positions 47 to 461 of SEQ ID NO: 16. FIX includes any form of FIX which has the typical features of FIX. Generally, FIX contains a GLA domain (a domain containing γ-carboxyglutamate residues), two EGF domains (human EGF homology domains), an activation peptide domain, and a C-terminal protease domain. However, FIX is not necessarily limited to FIX containing the above, and it may contain domains known in this technical field that are synonymous to these domains, or partially-deleted fragments thereof. FIX or sequence variants thereof are not limited to the following, but they have been cloned as described in U.S. Pat. Nos. 4,770,999 and 7,700,734, and cDNAs encoding human FIX have been isolated (see for example, Choo et al., Nature 299: 178-180 (1982); Fair et al., Blood 64: 194-204 (1984); and Kurachi et al., Proc. Natl. Acad. Sci., U.S.A. 79: 6461-6464 (1982)). These known sequence variants include those carrying amino acid substitutions that enhance the functions of FIX and amino acid substitutions that prolong the half-life of FIX. Furthermore, as long as the objective of the present invention can be accomplished, various FIX variants (for example, FIX into which amino acid sequence mutations are artificially introduced) and various modified forms of FIX (for example, PEGylated FIX) may be used. Herein below, the term “FIX” is simply mentioned in the present application it includes its sequence variants, FIX variants, and modified forms FIX, unless specifically noted otherwise.

As used herein, embodiments represented by the expression “comprising . . . ” may include embodiments represented by the expression “essentially consisting of . . . ” and embodiments represented by the expression “consisting of . . . ”.

All patents and reference documents explicitly cited herein are incorporated by reference into this description in their entirety.

The present invention will be further illustrated by the Examples, but it is not to be construed as being limited thereto.

### EXAMPLES

Herein below, the present invention will be specifically described by the Examples, but it is not to be construed as being limited thereto.

### Example 1

In the present invention, the blood/plasma procoagulant activity of a multispecific antigen-binding molecule that functionally substitutes for FVIII was examined using blood or plasma from FIX disorders other than hemophilia A, acquired hemophilia A, von Willebrand disease, and hemophilia C. More specifically, the blood/plasma procoagulant activity of ACE910 (Emicizumab), which is the bispecific antibody described in a patent document (WO 2012/067176) and is one of the aforementioned multispecific antigen-binding molecules, was examined by each of the coagulation evaluation methods, ROTEM and APTT, using blood/plasma derived from hemophilia B patients and a commercially-available FIX-deficient human plasma (George King Bio-Medical).

### Example 2

### Preparation of ACE910, which is an Anti-FIXa/FX Bispecific Antibody that Substitutes for FVIII Function

ACE910 was obtained by the methods described in WO 2005/035756, WO 2006/109592, and WO 2012/067176. The bispecific antibody was expressed by incorporating the antibody genes into animal cell expression vectors and transfecting them into CHO cells. The bispecific antibody contained in the cell culture supernatant was then purified.

The FVIII function-substituting activity of the thus purified bispecific antibody was measured by the enzyme assay shown below. At room temperature, 1 nM human FIXa (Enzyme Research Laboratories), 140 nM human FX (Enzyme Research Laboratories), 20 μM phospholipids (10% phosphatidylserine, 60% phosphatidylcholine, 30% phosphatidylethanolamine), and the bispecific antibody were mixed with Tris-buffered saline solution containing 5 mM CaCl₂) and 0.1% bovine serum albumin, and incubated for 2 minutes to cause the FX activation reaction by FIXa to proceed. This reaction was terminated by adding EDTA. Subsequently, FXa-specific chromogenic substrate solution S-2222 (CHROMOGENIX) was added, and the change in absorbance at 405 nm was measured using SpectraMax 340PC384 (Molecular Devices). A calibration curve was prepared from the change in absorbance by human FXa (Enzyme Research Laboratories) at known concentrations, and the FXa production-promoting activity of the bispecific antibody was evaluated.

### Example 3

### ROTEM Measurements

ROTEM measurements were performed according to a usual method using the ROTEM delta measurement device (Tem International GmbH). As a Ca trigger, a calcium solution star-tem reagent (Ref. No. 503-01, Tem International GmbH) was used.

### APTT Measurements

Thrombocheck APTT-SLA (Sysmex) was used as the APTT reagent. 50 μL of the APTT reagent was added to 50 μL of FIX disorder patient-derived plasma or FIX-deficient human plasma containing ACE910 and/or anti-FIX-Gla antibody (Thromb Res 2000; 100:73-79). After incubation at 37 °C for five minutes, 50 μL of 0.02 mol/L calcium chloride solution was added to initiate the coagulation reaction, and APTT was measured according to a conventional method with an automated blood coagulation measurement device (CS-2000i, Sysmex).

### Example 4

### Results of ROTEM Measurements

Using samples prepared by adding ACE910 to blood derived from patients with FIX disorder, percentage shortening of blood coagulation (initiation) time [(1-coagulation (initiation) time after addition of ACE910/coagulation (initiation) time before addition of ACE910)×100] according to Ca-triggered ROTEM was determined. Blood coagulation (initiation) time according to ROTEM was calculated as the sum of clotting time (CT) and clot formation time (CFT) measured by the ROTEM delta measurement device.

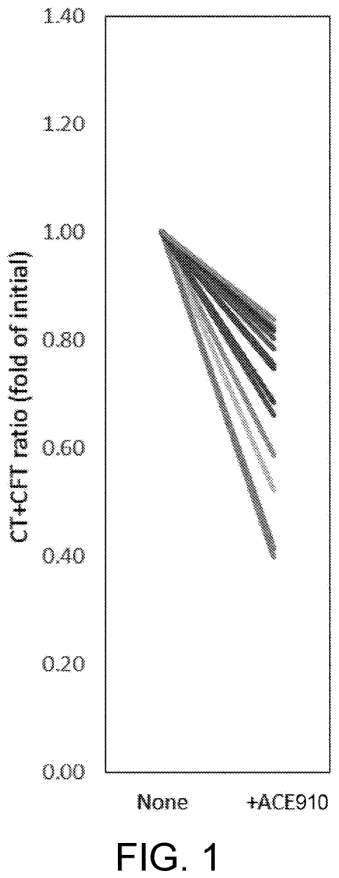

Ca-triggered ROTEM was performed using 25 blood samples derived from 17 cases of FIX disorder patients under regular administration therapy with an FIX formulation (median FIX:C of 1.9 IU/dL; and FIX:C range of less than 0.2 IU/dL to 16.5 IU/dL). As a result, the coagulation (initiation) time was shortened in most cases by ex vivo addition of ACE910 (50 μg/mL), with a median percentage shortening of 18% (FIG. 1 shows the ratios of blood coagulation (initiation) time for some of the samples).

### Results of APTT Measurements

Percentage shortening of APTT [(1−APTT after addition of ACE910/APTT before addition of ACE910)×100] was determined using samples prepared by adding ACE910 to plasma derived from FIX disorder patients or FIX-deficient human plasma.

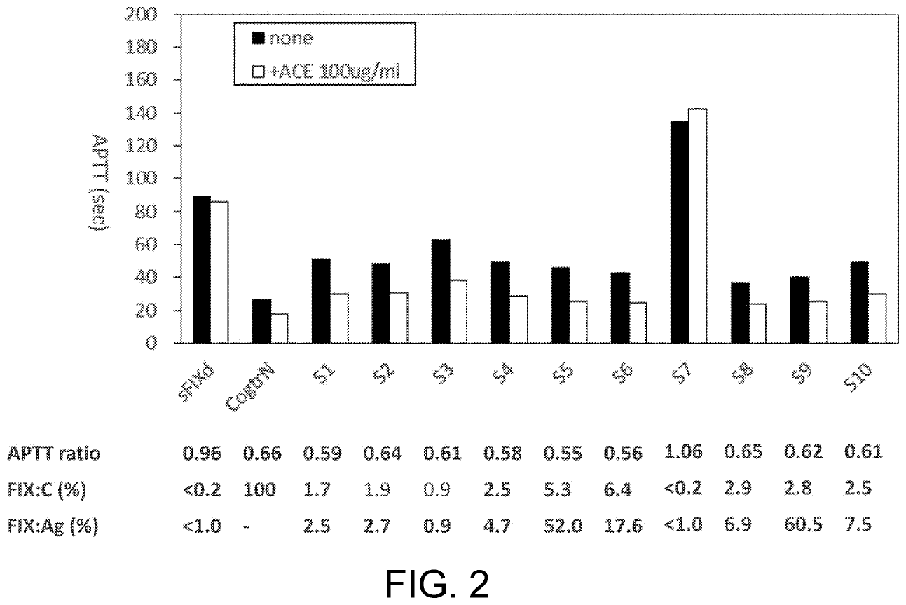

When ACE910 (100 μg/mL) was added ex vivo in the plasma of 10 cases of FIX disorder patients, no APTT shortening effect was observed in one FIX inhibitor-positive case, with a shortening ratio of −6% (APTT ratio of 1.06). However, in all of the remaining nine cases (FIX:C range of 0.9 IU/dL to 6.4 IU/dL), ACE910 was effective in shortening APTT, with a median shortening ratio of 39% (ranging from 35% to 45%) (APTT ratio of 0.55 to 0.65) (FIG. 2).

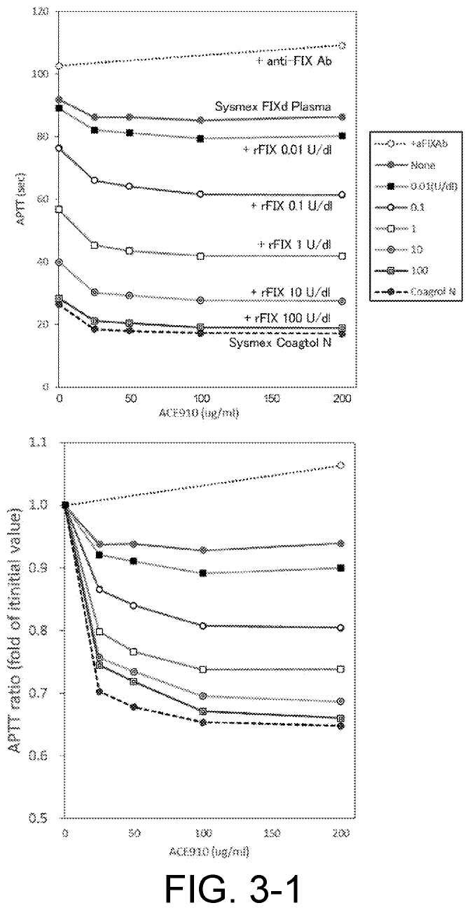

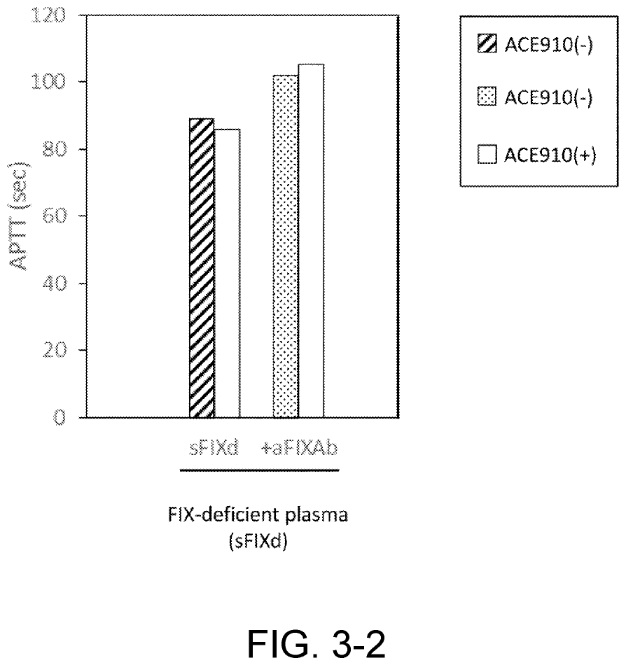

FIX formulation (BeneFix, Pfizer) was added at concentrations of 0, 0.01, 0.1, 1, 10, and 100 IU/dL to commercially-available FIX-deficient human plasma, and ACE910 was further added ex vivo. The percentage by which the APTT was shortened after addition of 100 μg/mL ACE910 was evaluated. The result showed that the percentage APTT shortening was 7%, 11%, 19%, 26%, 30%, and 33% according to the respective concentrations of the FIX formulation (FIG. 3-1). The APTT shortening effect of ACE910 was FIX concentration-dependent, and when the FIX concentration was from 1 IU/dL to 100 IU/dL, the level of change in the shortening ratio was nearly stable (26% to 33%). This effect was also slightly observed (shortening ratio of 7%) in FIX-deficient human plasma to which the FIX formulation was not added, but further addition of an anti-FIX-Gla antibody (anti-FIX Ab: prepared by referring to Thromb Res 2000; 100: 73-79) to this plasma ex vivo (anti-FIX-Gla antibody: 150 μg/mL) cancelled this effect (shortening ratio of −3%) (FIG. 3-2). For the ACE910-added groups, 100 g/mL ACE910 was added. Therefore, it was shown that FIX is essential for ACE910 to express its effect, and the effect is exerted in the presence of even a trace amount of FIX (at the level of 0.01 IU/dL). A calibration curve for APTT shortening by changes in the concentration of FIX was prepared by adding only the FIX formulation at varying concentrations to FIX-deficient human plasma, and this calibration curve was used to convert the APTT obtained with addition of ACE910 into FIX:C. As a result, the maximum effect of ACE910 in the presence of 0.1 IU/dL, 1 IU/dL and 10 IU/dL of FIX was equivalent to 0.6 IU/dL, 11 IU/dL, and 114 IU/dL of FIX:C (i.e., %), respectively. Thus, the effect of the FIX formulation was enhanced six to eleven times. Many severe hemophilia B cases have a little FIX activity of less than 1.0 IU/dL. This result suggested that ACE910 can be applied to hemophilia B patients with a very small amount of FIX.

### INDUSTRIAL APPLICABILITY

The present invention provides methods of using multispecific antigen-binding molecules that functionally substitute for FVIII as a prevention method and/or a treatment method for onset and/or progress of bleeding, diseases accompanying bleeding, or diseases caused by bleeding in FIX disorders other than hemophilia A, acquired hemophilia A, von Willebrand disease, and hemophilia C. It is considered that multispecific antigen-binding molecules that functionally substitute for FVIII can be used not only as prevention methods and/or treating methods for bleeding in hemophilia A, acquired hemophilia A, von Willebrand disease, and hemophilia C which are caused by FVIII dysfunction, but also as prevention methods and/or treating methods for bleeding in other FIX disorders, because of its procoagulant activity. In current therapy of FIX disorders, the need of repeated intravenous administration of a FIX formulation leads to the problem of difficulty in securing vascular access, and is very burdensome to the patients and care givers particularly in pediatric cases. The antibodies which are multispecific antigen-binding molecules that functionally substitute for FVIII (as an example, ACE910) have been developed as a formulation for subcutaneous administration, and are expected to reduce the burden of administration compared to FIX formulations which require repeated intravenous administration. Therefore, the present invention may be promising as a prophylactic agent and/or therapeutic agent for FIX disorders. Furthermore, combined use of a multispecific antigen-binding molecule that functionally substitutes for FVIII and a FIX formulation can enhance the effect of the FIX formulation, and this may be promising as a combination therapy which shows stable hemostatic effects.

1. A pharmaceutical composition for use in preventing and/or treating blood coagulation factor IX disorder, which comprises a multispecific antigen-binding molecule that functionally substitutes for blood coagulation factor VIII.

2. The pharmaceutical composition of claim 1, wherein the multispecific antigen-binding molecule that functionally substitutes for blood coagulation factor VIII is a bispecific antibody which recognizes blood coagulation factor IX and/or activated blood coagulation factor IX, and blood coagulation factor X.

3. The pharmaceutical composition of claim 2, wherein the bispecific antibody is:
a bispecific antibody in which a first polypeptide and a third polypeptide form a pair and a second polypeptide and a fourth polypeptide form a pair, wherein the first polypeptide consists of an H chain comprising the H chain CDR 1, 2, and 3 amino acid sequences of SEQ ID NOs: 1, 2, and 3 (H chain CDRs of Q499), the second polypeptide consists of an H chain comprising the H chain CDR 1, 2, and 3 amino acid sequences of SEQ ID NOs: 4, 5, and 6 (H chain CDRs of J327), and the third polypeptide and the fourth polypeptide consist of a common L chain comprising the L chain CDR 1, 2, and 3 amino acid sequences of SEQ ID NOs: 7, 8, and 9 (L chain CDRs of L404).

4. The pharmaceutical composition of claim 2 or 3, wherein the bispecific antibody is:
a bispecific antibody in which a first polypeptide and a third polypeptide form a pair and a second polypeptide and a fourth polypeptide form a pair, wherein the first polypeptide consists of an H chain comprising the H chain variable region amino acid sequence of SEQ ID NO: 13, the second polypeptide consists of an H chain comprising the H chain variable region amino acid sequence of SEQ ID NO: 14, and the third polypeptide and the fourth polypeptide consist of a common L chain comprising the L chain variable region amino acid sequence of SEQ ID NO: 15.

5. The pharmaceutical composition of any one of claims 2 to 4, wherein the bispecific antibody is:
a bispecific antibody in which a first polypeptide and a third polypeptide form a pair and a second polypeptide and a fourth polypeptide form a pair, wherein the first polypeptide consists of an H chain consisting of the amino acid sequence of SEQ ID NO: 10, the second polypeptide consists of an H chain consisting of the amino acid sequence of SEQ ID NO: 11, and the third polypeptide and the fourth polypeptide consist of a common L chain of SEQ ID NO: 12.

6. The pharmaceutical composition of any one of claims 1 to 5, wherein the blood coagulation factor IX disorder is a disease that develops and/or progresses due to a decrease, dysfunction, and/or defect in the activity of blood coagulation factor IX and/or activated blood coagulation factor IX.

7. The pharmaceutical composition of any one of claims 1 to 6, wherein the blood coagulation factor IX disorder is a congenital or acquired disease.

8. The pharmaceutical composition of any one of claims 1 to 7, wherein the blood coagulation factor IX disorder is hemophilia B or blood coagulation factor IX deficiency disease.

9. The pharmaceutical composition of any one of claims 1 to 8, which is for combined use with a blood coagulation factor IX formulation.

10. The pharmaceutical composition of any one of claims 1 to 8, which is for enhancing the blood coagulation activity of a blood coagulation factor IX formulation.

11. A combination medicament for use in preventing and/or treating blood coagulation factor IX disorder, which is a combination of a multispecific antigen-binding molecule that functionally substitutes for blood coagulation factor VIII and a blood coagulation factor IX formulation.

12. A method of enhancing the blood coagulation activity of a blood coagulation factor IX formulation in a patient with a blood coagulation factor IX disorder, using a multispecific antigen-binding molecule that functionally substitutes for blood coagulation factor VIII.

13. The method of claim 12, wherein the method prevents or reduces the frequency of bleeding episodes in the patient.

14. The method of claim 12, wherein the patient is treated prophylactically with the bispecific antibody, to prevent or to reduce the frequency of bleeding episodes in the patient.


SEQUENCE LISTING (16 sequences)

SEQ ID NO: 1 - protein, 5 aa, synthetic construct
  REGION 1..5: note = Heavy chain variable region CDR1
YYDIQ

SEQ ID NO: 2 - protein, 17 aa, synthetic construct
  REGION 1..17: note = Heavy chain variable region CDR2
SISPSGQSTYYRREVKG

SEQ ID NO: 3 - protein, 14 aa, synthetic construct
  REGION 1..14: note = Heavy chain variable region CDR3
RTGREYGGGWYFDY

SEQ ID NO: 4 - protein, 5 aa, synthetic construct
  REGION 1..5: note = Heavy chain variable region CDR1
DNNMD

SEQ ID NO: 5 - protein, 17 aa, synthetic construct
  REGION 1..17: note = Heavy chain variable region CDR2
DINTRSGGSIYNEEFQD

SEQ ID NO: 6 - protein, 10 aa, synthetic construct
  REGION 1..10: note = Heavy chain variable region CDR3
RKSYGYYLDE

SEQ ID NO: 7 - protein, 11 aa, synthetic construct
  REGION 1..11: note = Light chain variable region CDR1
KASRNIERQLA

SEQ ID NO: 8 - protein, 7 aa, synthetic construct
  REGION 1..7: note = Light chain variable region CDR2
QASRKES

SEQ ID NO: 9 - protein, 9 aa, synthetic construct
  REGION 1..9: note = Light chain variable region CDR3
QQYSDPPLT

SEQ ID NO: 10 - protein, 448 aa, synthetic construct
  REGION 1..448: note = Heavy chain
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 11 - protein, 444 aa, synthetic construct
  REGION 1..444: note = Heavy chain
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 12 - protein, 214 aa, synthetic construct
  REGION 1..214: note = Light chain
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 123 aa, synthetic construct
  REGION 1..123: note = Heavy chain variable region
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 14 - protein, 119 aa, synthetic construct
  REGION 1..119: note = Heavy chain variable region
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 15 - protein, 107 aa, synthetic construct
  REGION 1..107: note = Light chain variable region
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIK

SEQ ID NO: 16 - protein, 461 aa, Homo sapiens
  SIGNAL 1..46
  REGION 47..461: note = mat_peptide
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT